(19) United States
(12) Patent Application Publication
(10) Pub. No.: US 2025/0000844 A1
(43) Pub. Date: Jan. 2, 2025

(54) PET SHAMPOOS, PET FOOD COMPOSITIONS, AND REGIMENS AND/OR METHODS THEREOF

(71) Applicant: Hill's Pet Nutrition, Inc., Overland Park, KS (US)

(72) Inventors: Dayakar BADRI, Lawrence, KS (US); Sasha Marie Barnett, Lawrence, KS (US); Kimberly Marie Wilson, Lawrence, KS (US); Jose Bucio, Mexico City (MX); Claudia Bentosa, Azcapotzalco (MX)

(73) Assignee: Hill's Pet Nutrition, Inc., Overland Park, KS (US)

(21) Appl. No.: 18/756,828

(22) Filed: Jun. 27, 2024

Related U.S. Application Data
(60) Provisional application No. 63/524,059, filed on Jun. 29, 2023.

(51) Int. Cl. A61K 31/385; A23K 20/121; A23K 20/158; A23K 20/174; A23K 50/40; A61K 8/34; A61K 8/44; A61K 8/60; A61K 8/9789; A61K 31/352; A61K 31/355; A61P 17/08; A61P 17/14; A61Q 5/02

(52) U.S. Cl. CPC A61K 31/385; A23K 20/121; A23K 20/158; A23K 20/174; A23K 50/40; A61K 8/345; A61K 8/442; A61K 8/602; A61K 8/9789; A61K 31/352; A61K 31/355; A61P 17/08; A61P 17/14; A61Q 5/02; A61K 2800/596

(57) ABSTRACT

Pet shampoos, pet food compositions, and methods thereof are disclosed herein. In accordance with an aspect, provided is a method for reducing alopecia, skin secretion, ear secretion, and/or erythema scores of a companion animal comprising: feeding a pet food composition to a pet, and applying a pet shampoo to the pet within about 36 hours of providing the pet food composition to the pet, wherein the pet food composition comprises a plurality of fatty acids selected from omega-3 fatty acids, omega-6 fatty acids, omega-9 fatty acids, and a combination thereof, quercetin, and one or more vitamins comprising a vitamin C, a vitamin E, or a combination thereof; and wherein the pet shampoo comprises: from about 1 to about 15 wt. % of an amphoteric surfactant, from about 5 to about 25 wt. % of a nonionic surfactant, and from about 1 to about 20 wt. % of a polyol.

### CROSS-REFERENCE TO RELATED APPLICATIONS

This application claims the benefit of priority from U.S. Patent Application No. 63/524,059, filed Jun. 29, 2023, titled Pet Shampoos, Pet Food Compositions, and Regimens And/or Methods Thereof, the content of which are hereby incorporated herein in their entireties, for all purposes.

### BACKGROUND

Approximately 15-25% of all small animal veterinary practice activity is devoted to problems related to the skin and coat of dogs and cats. Dermatological disorders, such as alopecia along with skin and ear secretions can reduce the overall quality of a pet. For instance, pets suffering from dermatological disorders may exhibit behavioral patterns such as head shaking; scratching of cars, paws, armpits, and groin; overall redness of skin; and skin oiliness across the body.

In particular, canine atopic dermatitis (CAD) is a chronic inflammatory skin disease in canines. A combination of genetic, environmental, and immunological factors influences the pathogenesis of CAD and its outcome. CAD is characterized by pruritus (itchiness), erythema (redness), skin and car secretions and alopecia (hair loss). Pruritus and erythema are key characteristics of CAD and are a common reason for a veterinary visit. Besides genetic factors, food and environmental allergens are critical factors that contribute to skin allergic reactions in dogs. Environmental factors also induce skin allergic reactions in dogs that contribute to the development of CAD, specifically in genetically predisposed breeds.

In addition to a reduced quality of life, pets suffering from CAD often suffer from pruritus/itchy skin disorder and/or skin membrane barrier dysfunction. Skin barrier plays a critical role in preventing the entry of allergens and microorganisms into the body. The physical skin barrier is localized in the uppermost area of the epidermis called stratum corneum. Further, the epidermis is continuously regenerated by terminally differentiating keratinocytes, a process called keratinization or cornification. Cornification begins with the migration of keratinocytes from the basal to upper layers during which keratinocytes also produce lipids and extrude them into the extracellular space to form extra-cellular enriched layers to maintain skin membrane barrier function. In addition, the presence of cytokines within the skin influences various processes of keratinocyte proliferation, differentiation and cornification.

### BRIEF SUMMARY

This summary is intended merely to introduce some aspects of one or more implementations of the present disclosure. Further areas of applicability of the present disclosure will become apparent from the detailed description provided hereinafter. This summary is not an extensive overview, nor is it intended to identify key or critical elements of the present teachings, nor to delineate the scope of the disclosure. Rather, its purpose is merely to present one or more concepts in simplified form as a prelude to the detailed description below.

In accordance with an aspect of the invention, provided is a method for improving the health and/or appearance of a pet's skin and/or hair. The method generally comprises reducing alopecia, skin secretion, car secretion, and/or erythema scores of a pet by feeding a pet food composition to a pet, and applying a pet shampoo to the pet within about 36 hours of providing the pet food composition to the pet. The pet food composition may comprise a plurality of fatty acids selected from omega-3 fatty acids, omega-6 fatty acids, omega-9 fatty acids, and a combination of two or more thereof, quercetin, and one or more vitamins, the one or more vitamins comprising a vitamin C, a vitamin E, or a combination thereof. The pet shampoo may comprise from about 1 to about 15 wt. % of an amphoteric surfactant, from about 5 to about 25 wt. % of a nonionic surfactant, and from about 1 to about 20 wt. % of a polyol.

In accordance with another aspect of the invention, provided is a pet shampoo for enhancing the benefits of a pet food composition for reducing alopecia, skin secretions, car secretions, and/or erythema scores. The pet shampoo is typically adapted to enhance the reduction of alopecia, skin secretion, car secretion, and/or erythema scores and generally comprises a polyol; an amphoteric surfactant; and a nonionic surfactant, wherein the amphoteric surfactant and the nonionic surfactant are in a weight ratio of amphoteric surfactant to nonionic surfactant of about 1:1.5 to about 1:5.

A non-limiting list of embodiments of aspects of the invention is provided below:

In an embodiment 1, a method is provided for improving the health and/or appearance of a pet's skin and/or hair, the method comprising:
feeding a pet food composition to a pet, the pet food composition comprising:
   a plurality of fatty acids selected from omega-3 fatty acids, omega-6 fatty acids, omega-9 fatty acids, and a combination of two or more thereof;
   quercetin; and
   one or more vitamins, the one or more vitamins comprising a vitamin C, a vitamin E, or a combination thereof; and
applying a pet shampoo to the pet within about 36 hours of providing the pet food composition to the pet, the pet shampoo comprising:
   from about 1 to about 15 wt. % of an amphoteric surfactant;
   from about 5 to about 25 wt. % of a nonionic surfactant; and
   from about 1 to about 20 wt. % of a polyol.

In accordance with an embodiment 2, provided the method according to claim 1 further comprising: rinsing the pet after applying the pet shampoo to the pet, wherein the pet is rinsed after a period of time ranging from about 30 seconds to about 30 minutes after the pet shampoo is applied.

In accordance with an embodiment 3, provided the method according to claim 1 or claim 2 comprising reducing alopecia by about 20% or more, preferably about 30% or more, preferably about 35% or more, preferably about 40% or more, or preferably about 50% or more.

In accordance with an embodiment 4, provided the method according to any foregoing claim comprising reducing the skin secretions and/or the ear secretions of the pet by about 40% or more, preferably about 60% or more, or preferably about 70% or more.

In accordance with an embodiment 5, provided the method according to any foregoing claim comprising reducing erythema by about 50% or more, preferably about 70% or more, preferably about 80% or more, or preferably about 85% or more.

In accordance with an embodiment 6, provided the method according to any foregoing claim, wherein the amphoteric surfactant is selected from alkyl amphopropionates, betaines, alkyl sultaines, alkyl amphoacetates, and a combination of two or more thereof.

In accordance with an embodiment 7, provided the method according to claim 6, wherein the amphoteric surfactant comprises a betaine surfactant.

In accordance with an embodiment 8, provided the method according to claim 6 or claim 7, wherein the amphoteric surfactant comprises cocamidopropyl betaine.

In accordance with an embodiment 9, provided the method according to any foregoing claim, wherein the nonionic surfactant comprises a glucoside surfactant.

In accordance with an embodiment 10, provided the method according to claim 9, wherein the glucoside surfactant is selected from decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination of two or more thereof.

In accordance with an embodiment 11, provided the method according to any foregoing claim, wherein the pet shampoo has a weight ratio of the amphoteric surfactant to the nonionic surfactant of about 1:1.5 to about 1:5.

In accordance with an embodiment 12, provided the method according to any foregoing claim, wherein the polyol is selected from glycerin, 1,3-propanediol, isoprene glycol, pentylene glycol, hexylene glycol, ethylene glycol, propylene glycol, butylene glycol, diethylene glycol, dipropylene glycol, and a combination of two or more thereof.

In accordance with an embodiment 13, provided the method according to any foregoing claim, wherein the pet shampoo further comprises an oil.

In accordance with an embodiment 14, provided the method according to claim 13, wherein the oil is a natural oil.

In accordance with an embodiment 15, provided the method according to claim 14, wherein the natural oil is selected from flax seed oil, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, marula oil, evening primrose oil, olive oil, palm oil, palm kernel oil, Brazil nut oil, pecan nut oil, peach kernel oil, rapeseed oil, castor oil, sea buckthorn pulp oil, sea buckthorn kernel oil, sesame oil, soy bean oil, sunflower oil, grape seed oil, walnut oil, wild rose oil, wheat germ oil, coconut oil, and a combination of two or more thereof.

In accordance with an embodiment 16, provided the method according to any foregoing claim, wherein the pet shampoo is substantially free, or free, of an anionic surfactant.

In accordance with an embodiment 17, provided the method according to any foregoing claim, wherein the plurality of fatty acids comprises one or more omega-3 fatty acid, one or more omega-6 fatty acid, and one or more omega-9 fatty acid.

In accordance with an embodiment 18, provided the method according to claim 17, wherein the pet food composition has a ratio of the total amount of the one or more omega-3 fatty acid to the total amount of the one or more omega-6 fatty acid to the total amount of the one or more omega-9 fatty acid in an amount ranging from about 0.5:2:2 to about 3:5:5.

In accordance with an embodiment 19, provided the method according to any foregoing claim, wherein the one or more vitamins comprises a vitamin C and a vitamin E.

In accordance with an embodiment 20, provided the method according to any foregoing claim, wherein the pet shampoo further comprises sodium gluconate.

In accordance with an embodiment 21, provided the method according to any foregoing claim, further comprising reducing the number of times the pet licks itself by about 40% or more.

In accordance with an embodiment 22, provided the method according to any foregoing claim, further comprising reducing the amount of car scratching by the pet by about 20% or more.

In accordance with an embodiment 23, provided the method according to any foregoing claim, further comprising reducing the amount of head shaking by the pet by about 10% or more.

In accordance with an embodiment 24, provided is a pet shampoo comprising:
an amphoteric surfactant;
a nonionic surfactant,
   wherein the amphoteric surfactant and the nonionic surfactant are in a weight ratio of amphoteric surfactant to nonionic surfactant of about 1:1.5 to about 1:5; and a polyol,
wherein the pet shampoo enhances the skin benefits provided by a pet food composition.

In accordance with an embodiment 25, provided the pet shampoo according to claim 24, wherein the skin benefit is selected from reduced alopecia; reduced skin secretions; reduced car secretions; erythema; and a combination of two or more thereof.

In accordance with an embodiment 26, provided the pet shampoo according to claim 24 or claim 25, wherein the amphoteric surfactant comprises cocamidopropyl betaine.

In accordance with an embodiment 27, provided the pet shampoo according to any one of claims 24 to 26, wherein the nonionic surfactant comprises a glucoside surfactant.

In accordance with an embodiment 28, provided the pet shampoo according to any one of claims 24 to 27, wherein the pet shampoo further comprises a natural oil.

In accordance with an embodiment 29, provided the pet shampoo according to claim 28, wherein the natural oil is selected from flax seed oil, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, marula oil, evening primrose oil, olive oil, palm oil, palm kernel oil, Brazil nut oil, pecan nut oil, peach kernel oil, rapeseed oil, castor oil, sea buckthorn pulp oil, sea buckthorn kernel oil, sesame oil, soy bean oil, sunflower oil, grape seed oil, walnut oil, wild rose oil, wheat germ oil, coconut oil, and a combination of two or more thereof.

In accordance with an embodiment 30, provided the pet shampoo according to any one of claims 24 to 29, wherein the pet shampoo is substantially free, or free, of an anionic surfactant.

In accordance with an embodiment 31, provided the pet shampoo according to any one of claims 24 to 30, further comprising sodium gluconate.

In accordance with an embodiment 32, a method is provided for treating, preventing or ameliorating a symptom associated with:
alopecia;
excessive skin secretions;
excessive ear secretions;
dermatitis; and/or erythema, in a companion animal in need thereof, comprising:
   feeding a pet food composition to the companion animal, the pet food composition comprising:
      a plurality of fatty acids selected from omega-3 fatty acids, omega-6 fatty acids, omega-9 fatty acids, and a combination of two or more thereof;
      quercetin;
      one or more vitamins, the one or more vitamins comprising a vitamin C, a vitamin E, or a combination thereof; and
   applying a pet shampoo to the pet within about 36 hours of providing the pet food composition to the pet, the pet shampoo comprising:
      from about 1 to about 15 wt. % of an amphoteric surfactant;
      from about 5 to about 25 wt. % of a nonionic surfactant; and
      from about 1 to about 20 wt. % of a polyol.

In accordance with an embodiment 33, aa method is provided for improving the health and/or appearance of a pet's skin and/or hair, the method comprising:
applying a pet shampoo to a pet, the pet shampoo comprising:
   from about 1 to about 15 wt. % of an amphoteric surfactant;
   from about 5 to about 25 wt. % of a nonionic surfactant; and
   from about 1 to about 20 wt. % of a polyol; and
feeding a pet food composition to the pet within about 36 hours of applying the pet shampoo to the pet, the pet food composition comprising:
   a plurality of fatty acids selected from omega-3 fatty acids, omega-6 fatty acids, omega-9 fatty acids, and a combination of two or more thereof;
   quercetin; and
   one or more vitamins, the one or more vitamins comprising a vitamin C, a vitamin E, or a combination thereof.

### BRIEF DESCRIPTION OF THE DRAWINGS

The features, and advantages of the invention will be apparent from the following more detailed description of certain embodiments of the invention and as illustrated in the accompanying drawings in which:

FIG. 1 is a bar graph showing the reduction in alopecia, skin and ear secretions, and erythema scores for canines receiving the combination of an example pet food composition and an example pet shampoo when compared with a control group of canines in accordance with an aspect of the invention;

FIG. 2 is a bar graph indicating the improvement in skin membrane barrier function for canines receiving the combination of an example pet food composition and an example pet shampoo when compared with a control group of canines in accordance with an aspect of the invention;

FIG. 3 is a bar graph of the sensory evaluation of an example pet shampoo for “aroma” in accordance with an aspect of the invention; and

FIG. 4 is a bar graph of the sensory evaluation of the example pet shampoo of FIG. 3 for “overall likeness” in accordance with an aspect of the invention.

It should be understood that the various aspects are not limited to the compositions, and arrangements shown in the figures.

### DETAILED DESCRIPTION

For illustrative purposes, the principles of the present invention are described by referencing various exemplary embodiments thereof. Although certain embodiments of the invention are specifically described herein, one of ordinary skill in the art will readily recognize that the same principles are equally applicable to, and can be employed in other compositions and methods. Before explaining the disclosed embodiments of the present invention in detail, it is to be understood that the invention is not necessarily limited in its application to the details of any particular embodiment disclosed. The terminology used herein is for the purpose of description and not of limitation.

As used herein and in the appended claims, the singular forms “a”, “an”, and “the” include plural references unless the context dictates otherwise. The singular form of any class of the ingredients refers not only to one ingredient within that class, but also to a mixture of those ingredients. The terms “a” (or “an”), “one or more” and “at least one” may be used interchangeably herein. The terms “comprising”, “including”, and “having” may be used interchangeably. The term “include” should be interpreted as “include, but are not limited to”. The term “including” should be interpreted as “including, but are not limited to”.

As used throughout, ranges are used as shorthand for describing each and every value that is within the range. Any value within the range can be selected as the terminus of the range. Thus, a range from 1-5, includes specifically 1, 2, 3, 4 and 5, as well as subranges, such as 2-5, 3-5, 2-3, 2-4, 1-4, etc. The term “about” when referring to a number means any number within a range of 10% of the number. For example, the phrase “about 2 wt. %” refers to a number between and including 1.8 wt. % and 2.2 wt. %.

All references cited herein are hereby incorporated by reference in their entireties. In the event of a conflict in a definition in the present disclosure and that of a cited reference, the present disclosure controls.

The abbreviations and symbols as used herein, unless indicated otherwise, take their ordinary meaning. The abbreviation “wt. %” means wt. % with respect to the pet food composition. The symbol “°” refers to a degree, such as a temperature degree or a degree of an angle. The symbols “h”, “min”, “mL”, “nm”, “μm” means hour, minute, milliliter, nanometer, and micrometer, respectively. The abbreviation “UV-VIS” referring to a spectrometer or spectroscopy, means Ultraviolet-Visible. The abbreviation “rpm” means revolutions per minute.

Any member in a list of species that are used to exemplify or define a genus, may be mutually different from, or overlapping with, or a subset of, or equivalent to, or nearly the same as, or identical to, any other member of the list of species. Further, unless explicitly stated, such as when reciting a Markush group, the list of species that define or exemplify the genus is open, and it is given that other species may exist that define or exemplify the genus just as well as, or better than, any other species listed.

All components and elements positively set forth in this disclosure can be negatively excluded from the claims. In other words, the pet food compositions of the instant disclosure can be free or essentially free of all components and elements positively recited throughout the instant disclosure. In some instances, the pet food compositions of the present disclosure may be substantially free of non-incidental amounts of the ingredient(s) or compound(s) described herein. A non-incidental amount of an ingredient or compound is the amount of that ingredient or compound that is added into the pet food composition by itself. For example, a pet food composition may be substantially free of a non-incidental amount of an ingredient or compound, although such ingredient(s) or compound(s) may be present as part of a raw material that is included as a blend of two or more compounds. Substantially free, unless other defined or described herein, typically refers to an ingredient or compound in an amount of about 2 wt. % or less, about 1.5 wt. % or less, about 1 wt. % or less, about 0.5 wt. % or less, about 0.1 wt. % or less, or about 0.05 wt. % or less, or about 0.01 wt. % or less, based on the total weight of the pet food composition on a dry matter basis.

Some of the various categories of components identified may overlap. In such cases where overlap may exist and the pet food composition includes both components (or the composition includes more than two components that overlap), an overlapping compound does not represent more than one component. For example, certain components or ingredients may be characterized as both an ancient grain and an amaranth. If a particular pet food care composition recites both an ancient grain and an amaranth, a compound that may be characterized as both an ancient grain and an amaranth will serve only as either an ancient or an amaranth—not both.

As used herein, the term “pet” could be used interchangeably with “companion animal” and refers to an animal of any species kept by a caregiver as a pet or any animal of a variety of species that have been widely domesticated as pets, including canines (Canis familiaris) and felines (Felis domesticus). Thus, a pet may include but is not limited to, working dogs, pet dogs, cats kept for rodent control (i.e., farm cats), pet cats, ferrets, birds, reptiles, rabbits, and fish.

To the extent that the pet food composition and/or an ingredient therein contains water/moisture, the dry matter represents everything in the sample other than water including, for example, protein, fiber, fat, carbohydrates, minerals, etc. Dry matter weight is the total weight minus the weight of any water. The skilled artisan would readily recognize and understand nutritional amounts and percentages expressed as dry matter amounts, dry matter weights and dry matter percentages.

Dry matter intake per day is calculated as the total nutritional intake per day excluding all water. For example, an amount of an ingredient equal to a specific percent of daily nutritional intake refers to the amount of that ingredient in dry matter form (i.e., excluding all water) relative to the total amount of dry matter consumed (also excluding all water) in a day.

“Daily nutritional intake” and “total nutritional intake per day” refer to dry matter intake per day. That is, water weight is not included in calculating the amount of nutrition consumed per day. To calculate percent of an ingredient of total daily intake on a dry matter basis, water is removed from the total intake to give total daily dry matter intake and the percent of the ingredient is based on the amount of the ingredient present as dry matter.

As used herein, an “ingredient” refers to any component of a pet food composition. The term “nutrient” refers to a substance that provides nourishment and thus has a nutrient value. In some cases, an ingredient may comprise more than one “nutrient,” for example, a composition may comprise corn comprising important nutrients including both protein and carbohydrate.

Aspects of the invention are directed to pet food compositions, pet shampoos, and methods thereof. The methods and/or regimens disclosed herein typically employ certain pet shampoos that are specifically adapted for use in conjunction with certain pet food compositions to provide improvements to a pet's coat and skin. For instance, according to an aspect of the invention, provided is a method for improving the health and/or appearance of a pet's skin and/or hair. The method generally comprises reducing alopecia, skin secretion, car secretion, and/or erythema scores of a pet by feeding a pet food composition to a pet, and applying a pet shampoo to the pet within about 36 hours of providing the pet food composition to the pet. The pet food composition typically comprises a plurality of fatty acids selected from omega-3 fatty acids, omega-6 fatty acids, omega-9 fatty acids, and a combination of two or more thereof, quercetin, and one or more vitamins, the one or more vitamins comprising a vitamin C, a vitamin E, or a combination thereof. The pet shampoo may comprise from about 1 to about 15 wt. % of an amphoteric surfactant, from about 5 to about 25 wt. % of a nonionic surfactant, and from about 1 to about 20 wt. % of a polyol.

The methods and/or regimens disclosed herein may preferably employ a pet shampoo for enhancing the benefits of a pet food composition for reducing alopecia, skin secretions, car secretions, and/or erythema scores. The pet shampoo is typically adapted to enhance the reduction of alopecia, skin secretion, car secretion, and/or erythema scores and generally comprises a polyol; an amphoteric surfactant; and a nonionic surfactant, wherein the amphoteric surfactant and the nonionic surfactant are in a weight ratio of amphoteric surfactant to nonionic surfactant of about 1:1.5 to about 1:5.

The inventors were surprised that methods and/or regimens employing certain pet food compositions and certain pet shampoos achieved enhanced reductions in alopecia, skin and car secretion, and/or erythema. For example, the methods and/or regimens, employing embodiments of pet food compositions and pet shampoos disclosed herein, can achieve a reduction in the alopecia of a pet by about 20% or more, e.g., as assessed about 24 hours after a pet is washed with the pet shampoos by a pet consuming a recommended daily nutritional intake of the pet food composition and receiving a bi-weekly routine of washing with the pet shampoo for about a month. In some embodiments, the method may reduce the alopecia exhibited by the pet by about 30% or more, about 40% or more, about 45% or more, about 50% or more, about 55% or more, or about 60% or more, e.g., as assessed about 24 hours after a pet is washed with the pet shampoos by a pet consuming a recommended daily nutritional intake of the pet food composition and receiving a bi-weekly routine of washing with the pet shampoo for about a month.

Additionally or alternatively, methods and/or regimens, employing embodiments of pet food compositions and pet shampoos disclosed herein, can provide a reduction in the skin and car secretion of a pet by about 40% or more, e.g., as assessed about 24 hours after a pet is washed with the pet shampoo by a pet consuming a recommended daily nutritional intake of the pet food composition and receiving a bi-weekly routine of washing with the pet shampoo for about a month. For instance, the method may reduce the skin and car secretion of a pet by about 60% or more, about 65% or more, about 70% or more, about 75% or more, or about 80%, or more, e.g., as assessed about 24 hours after a pet is washed with the pet shampoos by a pet consuming a recommended daily nutritional intake of the pet food composition and receiving a bi-weekly routine of washing with the pet shampoo for about a month.

The methods and/or regimens, employing embodiments of pet food compositions and pet shampoos as disclosed herein, may preferably provide a reduction in the erythema of a pet by about 50% or more, e.g., as assessed about 24 hours after a pet is washed with the pet shampoos by a pet consuming a recommended daily nutritional intake of the pet food composition and receiving a bi-weekly routine of washing with the pet shampoo for about a month. In some embodiments, the method may reduce the erythema exhibited by the pet by about 60% or more, about 70% or more, about 75% or more, about 80% or more, or about 85% or more, e.g., as assessed about 24 hours after a pet is washed with the pet shampoos by a pet consuming a recommended daily nutritional intake of the pet food composition and receiving a bi-weekly routine of washing with the pet shampoo for about a month.

The methods and/or regimens, employing embodiments of pet food compositions and pet shampoos as disclosed herein, may preferably provide a reduction of the amount a pet licks itself by about 30% or more, e.g., about 35% or more, about 40% or more, about 42% or more, or about 45% or more, e.g., as assessed about 24 hours after a pet is washed with the pet shampoos by a pet consuming a recommended daily nutritional intake of the pet food composition and receiving a bi-weekly routine of washing with the pet shampoo for about a month. In some instances, the methods and/or regimens, employing embodiments of pet food compositions and pet shampoos as disclosed herein, provide a reduction in the amount of ear scratching by the pet by about 10% or more, about 15% or more, about 20% or more, about 22% or more, or about 25% or more, e.g., as assessed about 24 hours after a pet is washed with the pet shampoos by a pet consuming a recommended daily nutritional intake of the pet food composition and receiving a bi-weekly routine of washing with the pet shampoo for about a month. Additionally or alternatively, methods and/or regimens, employing embodiments of pet food compositions and pet shampoos as disclosed herein, may preferably provide a reduction in the amount of head shaking by about 7% or more, about 10% or more, about 12% or more, about 14% or more, or about 16% or more, e.g., as assessed about 24 hours after a pet is washed with the pet shampoos by a pet consuming a recommended daily nutritional intake of the pet food composition and receiving a bi-weekly routine of washing with the pet shampoo for about a month.

The methods and/or regimens may further comprise rinsing the pet after applying the pet shampoo to the pet. The pet may be rinsed after a period of time ranging from about 30 seconds to about 30 minutes after the pet shampoo is applied. In some embodiments, the pet is rinsed after a period of time of about 30 seconds to about 20 minutes, about 30 seconds to about 15 minutes, about 30 seconds to about 10 minutes, about 30 seconds to about 5 minutes, about 30 seconds to about 3 minutes, about 30 seconds to about 2 minutes, or about 30 seconds to about 1 minute, after the pet shampoo is applied.

The method may comprise washing the pet once a week, twice a week, or once every week for about a month or more, about 1.5 months or more, about 2 months or more, about 2.5 months or more, about 3 months or more, or any range or subrange thereof.

Suitable components, such as those listed below, may be included or excluded from the formulations for the pet food compositions or pet shampoos depending on the specific combination of other ingredients and the form of the pet food compositions or pet shampoos. In some embodiments, the pet food compositions disclosed herein may be in the form of a stand-alone pet food, as a supplement to pet food, as a pet food treat, or the like. The pet shampoo may be in the form of a pet shampoo, pet body wash, pet soap, and the like.

The pet shampoos disclosed herein typically comprise one or more amphoteric surfactant. The amphoteric surfactant may comprise a substituent containing 8 to 18 carbon atoms and a substituent containing one or more carboxylate, sulfonate, sulfate, phosphate, or phosphonate. For instance, the amphoteric surfactant may have an alkyl group comprising from 8 to 20 carbon atoms, 8 to 16 carbon atoms, 10 to 16 carbon atoms, or 10 to 13 carbon atoms. The amphoteric surfactant(s) may include, but are not limited to, derivatives of aliphatic secondary and tertiary amines in which the aliphatic radical can be straight chain or branched. In some cases, one of the aliphatic substituents of the amphoteric surfactant contains about 8 to about 18 carbon atoms and one of the aliphatic substituents contains an anionic water solubilizing group, e.g., carboxy, sulfonate, sulfate, phosphate, or phosphonate. The amphoteric surfactants disclosed herein may be present in the pet shampoo in a salt form.

The amphoteric surfactants may include alkyl amphopropionates, betaines, alkyl sultaines, alkyl amphoacetates, or a combination of two or more thereof. Preferably, the pet shampoo includes an amphoteric surfactant selected from betaine surfactants (also referred to herein as betaines). Examples of betaine surfactants include, e.g., alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl) carboxy methyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine. In some instances, the betaine surfactant is selected from coca betaine, cocamidopropyl betaine, lauryl betaine, laurylhydroxy sulfobetaine, lauryldimethyl betaine, behenyl betaine, capryl/capramidopropyl betaine, lauryl hydroxysultaine, stearyl betaine, and a combination of two or more thereof. For instance, the betaine surfactant may be coco betaine, cocamidopropyl betaine, behenyl betaine, capryl/capramidopropyl betaine, and lauryl betaine, or a combination of two or more thereof. In at least one embodiment, the pet shampoo comprises coco betaine, cocamidopropyl betaine, or a combination thereof.

The one or more amphoteric surfactant(s) may be present in the pet shampoo in an amount from about 1 to about 15 wt. %, based on the total weight of the pet shampoo. For example, the one or more amphoteric surfactant(s) may be present in a total amount of about 1 to about 15 wt. %, about 1 to about 12 wt. %, about 1 to about 9 wt. %, about 1 to about 6 wt. %, about 1 to about 4 wt. %; from about 3 to about 15 wt. %, about 3 to about 12 wt. %, about 3 to about 9 wt. %, about 3 to about 6 wt. %; from about 5 to about 15 wt. %, about 5 to about 12 wt. %, about 5 to about 9 wt. %; from about 7 to about 15 wt. %, about 7 to about 12 wt. %, about 7 to about 9 wt. %; from about 9 to about 15 wt. %, about 9 to about 12 wt. %, about 11 to about 15 wt. %, or any range or subrange thereof, based on the total weight of the pet shampoo.

The pet shampoos typically also comprises a nonionic surfactant. The nonionic surfactant may be selected from alkyl polyglucosides, alkylamides, alkanolamides, polyoxyalkylenated nonionic surfactants, polyglycerolated nonionic surfactants, ethoxylated fatty esters (preferably, polyglyceryl-2-caprate), and a combination of two or more thereof. The nonionic surfactant, such as alkyl polyglucosides, alkylamides, and alkanolamides, may have an alkyl group having 8 to 32 carbons, 8 to 26 carbons, 8 to 22 carbons, 8 to 18 carbons, 10 to 22 carbons, 10 to 18 carbons, 12 to 22 carbons, or 12 to 18 carbons. In at least one embodiment, the nonionic surfactant is an alkyl polyglucoside, an alkylamide, or an alkanolamide having an alkyl group of 12 to 18 carbons.

Examples of alkyl glucosides and/or alkyl polyglucosides include C₆₋₂₅ alkyl glucoside, e.g., C₈₋₁₈ alkyl glucoside, C₁₀₋₁₈ alkyl glucoside or C₁₀₋₁₆ alkyl glucoside, optionally wherein the alkyl glucoside is selected from decyl glucoside, caprylyl/capryl glucoside, lauryl glucoside, coco-glucoside, octyl glucoside, cetearyl glucoside, cetyl glucoside, hexadecyl glucoside, and arachidyl glucoside. The alkyl glucoside may be selected from decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination thereof.

Examples of alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one-OH group). Preferably, the fatty acid alkanolamides are chosen from fatty acid dicthanolamides (DEA) or fatty acid monoethanolamides (MEA), fatty acid monoisopropanolamides (MIPA), fatty acid diisopropanolamides (DIPA), and fatty acid glucamides (acyl glucamides). In some preferred embodiments, the nonionic surfactant comprises cocamide monoethanolamine (MEA), cocamide diethanolamine (DEA) or cocamide diisopropanolamine (DIPA), or a combination of two or more thereof. In at least one embodiment, the nonionic surfactant comprises cocamide DIPA.

Additional examples of fatty acid alkanolamides include oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monocthanolamide, isostearic acid monoisopropanolamide (isostearamide MIPA), crucic acid diethanolamide, ricinoleic acid monocthanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monocthanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

The nonionic surfactants may be selected from ethoxylated fatty esters, such as adducts of ethylene oxide with esters of lauric acid, palmitic acid, stearic acid or behenic acid, and mixtures thereof, including those containing from 9 to 100 oxyethylene groups. For example, the ethoxylated fatty esters comprise PEG-9 laurate to PEG-50 laurate; PEG-9 palmitate to PEG-50 palmitate; PEG-9 stearate to PEG-50 stearate; PEG-9 palmitostearate to PEG-50 palmitostearate; PEG-9 behenate to PEG-50 behenate; polyethylene glycol 100 EO monostearate (such as, PEG-100 stearate); and combinations of two or more thereof. As examples of glyceryl esters of fatty acids, glyceryl stearate (glyceryl mono-, di- and/or tristearate), glyceryl ricinoleate, and a combination of two or more thereof may be incorporated in the pet shampoo. Examples of glyceryl esters of C₈-C₂₄ alkoxylated fatty acids include polyethoxylated glyceryl stearate (glyceryl mono-, di- and/or tristearate), such as PEG-20 glyceryl stearate.

The nonionic surfactant may comprise a lactylate surfactant having carbon chains between C₈ and C₁₆, such as sodium lauroyl lactylate. Examples of lactylate surfactants include sodium decanoyl/dodecanoyl lactylate, tea-lauroyl lactylate, sodium hexanoyl lactylate, sodium behenoyl lactylate, sodium oleoyl lactylate, sodium decanoyl lactylate, sodium cocoyl lactylate, sodium lauroyl lactylate, sodium isostearoyl lactylate, calcium stearoyl lactylate, sodium stearoyl lactylate, or a combination of two or more thereof.

The pet shampoos may include one or more nonionic surfactant(s) in an amount that ranging from about 5 to about 25 wt. %, based on the total weight of the pet shampoo. For example, the total amount of nonionic surfactant(s) in the pet shampoo may be from about 5 to about 25 wt. %, about 5 to about 20 wt. %, about 5 to about 16 wt. %, about 5 to about 12 wt. %, about 5 to about 10 wt. %, about 5 to about 8 wt. %; from about 8 to about 25 wt. %, about 8 to about 20 wt. %, about 8 to about 16 wt. %, about 8 to about 12 wt. %; from about 11 to about 25 wt. %, about 11 to about 20 wt. %, about 11 to about 16 wt. %; from about 14 to about 25 wt. %, about 14 to about 21 wt. %, about 14 to about 17 wt. %; from about 17 to about 25 wt. %, about 17 to about 22 wt. %, about 17 to about 19 wt. %, about 20 to about 25 wt. %, or any range or subrange thereof, based on the total weight of the pet shampoo.

The pet shampoos may in certain embodiments be formulated to have a weight ratio of amphoteric surfactant to nonionic surfactant of about 1:1.5 to about 1:5. For instance, the pet shampoo may have a weight ratio of amphoteric surfactant to nonionic surfactant from about 1:1.5 to about 1:5, about 1:2 to about 1:5, about 1:3 to about 1:5, about 1:4 to about 1:5; from about 1:1.5 to about 1:4, about 1:2 to about 1:4, about 1:3 to about 1:4; from about 1:1.5 to about 1:3, about 1:2 to about 1:3; from about 1:1.5 to about 1:2.5, about 1:2 to about 1:2.5, about 1:1.5 to about 1:2, or any range or subrange thereof.

The pet shampoo may include one or more polyol(s). Typically, the pet shampoos include polyol(s) in an amount from about 3 to about 25 wt. %, based on the total weight of the pet shampoo. For example, the total amount of polyol(s) in the pet shampoo may be from about 3 to about 25 wt. %, about 3 to about 22 wt. %, about 3 to about 20 wt. %, about 3 to about 18 wt. %, about 3 to about 16 wt. %; from about 5 to about 25 wt. %, about 5 to about 22 wt. %, about 5 to about 20 wt. %, about 5 to about 18 wt. %, about 5 to about 16 wt. %; from about 7 to about 25 wt. %, about 7 to about 22 wt. %, about 7 to about 20 wt. %, about 7 to about 18 wt. %, about 7 to about 16 wt. %; from about 9 to about 25 wt. %, about 9 to about 22 wt. %, about 9 to about 20 wt. %, about 9 to about 18 wt. %, about 9 to about 16 wt. %; from about 12 to about 25 wt. %, about 12 to about 22 wt. %, about 12 to about 20 wt. %, about 12 to about 18 wt. %, about 12 to about 16 wt. %; from about 14 to about 25 wt. %, about 14 to about 22 wt. %, about 14 to about 20 wt. %, about 14 to about 18 wt. %; from about 16 to about 25 wt. %, about 16 to about 22 wt. %, about 16 to about 20 wt. %; from about 18 to about 25 wt. %, about 21 to about 25 wt. %, or any range or subrange thereof, based on the total weight of the pet shampoo.

The polyol(s) may be chosen from glycols or compounds with numerous hydroxyl groups. In some cases, the one or more polyols is/are selected from the group consisting of C₂-C₃₂ polyols. The one or more polyols may be liquid at ambient temperature (25° C.). The one or more polyols may have from 2 to 32 carbon atoms, from 3 to 16 carbon atoms, or from 3 to 12 carbon atoms.

Polyols that may be included in the pet shampoo, in certain instances, include ethylene glycol, propylene glycol, butylene glycol, hexylene glycol, glycerin, diglycerin, diethylene glycol, and dipropylene glycol, and mixtures thereof. In some cases, the polyol is propylene glycol. In some further cases, the polyol is one or both of propylene glycol and butylene glycol. Additionally, in some cases, the pet shampoo comprises at least propylene glycol, and optionally one or more polyols other than propylene glycol.

Non-limiting examples of polyols that may, optionally, be included in the pet wash include and/or may be chosen from alkanediols such as glycerin, 1,2,6-hexanetriol, trimethylolpropane, ethylene glycol, propylene glycol, butylene glycol, pentylene glycol, hexylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, dipropylene glycol mono-iso-propyl ether, sorbitol, sorbitan, triacetin, and a mixture thereof.

The one or more polyols may, optionally, be glycols or glycol ethers such as, e.g., monomethyl, monoethyl and monobutyl ethers of ethylene glycol, propylene glycol or ethers thereof such as, e.g., monomethyl ether of propylene glycol, butylene glycol, hexylene glycol, dipropylene glycol as well as alkyl ethers of diethylene glycol, e.g., monoethyl ether or monobutyl ether of diethylene glycol. In some cases, the one or more polyols may include or are chosen from ethylene glycol, propylene glycol, butylene glycol, hexylene glycol, pentylene glycol, 1,3-propanediol, diethylene glycol, dipropylene glycol, 1,4-butanediol, 1,5-pentanediol, hexane-1,6-diol, glycerin, diglycerin, caprylyl glycol, and a mixture thereof.

In certain embodiments, the pet shampoo may include polyol(s) selected from glycerin (preferably, vegetable refined glycerin), butylene glycol, and 1,3-propanediol, polyglutamic acid, saccharide isomerate, and combinations thereof. In some embodiments, the polyol is a polyethylene glycol, such as polyethylene glycol 600 (CAS-25322-68-3). The polyol may comprise ethylene glycol, propylene glycol, butylene glycol, hexylene glycol, glycerin, diglycerin, diethylene glycol, and dipropylene glycol, or a combination of two more thereof.

One or more oils may be included in the pet shampoo. The one or more oils may be selected synthetic oils and/or plant-based oils. Examples of plant-based oils include alexandria laurel tree oil, avocado oil, apricot stone oil, barley oil, borage seed oil, calendula oil, canelle nut tree oil, canola oil, caprylic/capric triglyceride castor oil, coconut oil, corn oil, cotton oil, cottonseed oil, evening primrose oil, flaxseed oil, groundnut oil, hazelnut oil, glycereth triacetate, glycerol triheptanoate, glyceryl trioctanoate, glyceryl triundecanoate, hempseed oil, jojoba oil, lucerne oil, maize germ oil, marrow oil, millet oil, neopentylglycol dicaprylate/dicaprate, olive oil, palm oil, passionflower oil, pentaerythrityl tetrastearate, poppy oil, propylene glycol ricinoleate, rapeseed oil, rye oil, safflower oil, sesame oil, shea butter, soya oil, soybean oil, sweet almond oil, sunflower oil, sysymbrium oil, syzigium aromaticum oil, tea tree oil, walnut oil, wheat germ glycerides and wheat germ oil. As examples of linear alkanes that may be included in the pet shampoo, mention may be made of n-heptane (C₇), n-octane (C₈), n-nonane (C₉), n-decane (C₁₀), n-undecane (C₁₁), n-dodecane (C₁₂), n-tridecane (C₁₃) and n-tetradecane (C₁₄), and mixtures thereof. According to a particular embodiment, the volatile linear alkane is chosen from n-nonane, n-undecane, n-dodecane, n-tridecane and n-tetradecane, and a combination of two or more thereof.

The amount of oil(s) present in the pet shampoo may range from about 0.1 to about 5 wt. %, based on the total weight of the pet shampoo. For example, the one or more oil(s) may be present in the pet shampoo in an amount from about 0.1 to about 4 wt. %, about 0.1 to about 3 wt. %, about 0.1 to about 2 wt. %, about 0.1 to about 1 wt. %; from about 0.3 to about 5 wt. %, about 0.3 to about 4 wt. %, about 0.3 to about 3 wt. %, about 0.3 to about 2 wt. %, about 0.3 to about 1 wt. %; from about 0.6 to about 5 wt. %, about 0.6 to about 4 wt. %, about 0.6 to about 3 wt. %, about 0.6 to about 2 wt. %, about 0.6 to about 1 wt. %; from about 1 to about 5 wt. %, about 1 to about 4 wt. %, about 1 to about 3 wt. %, about 1 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, including any range or subrange thereof, based on the total weight of the pet shampoo.

The pet shampoo may optionally include one or more thickening agent(s). The amount of thickening agent in the pet shampoo, if present, may be from about 0.1 to about 7 wt. %, about 0.1 to about 5 wt. %, about 0.1 to about 4 wt. %, about 0.1 to about 3 wt. %, about 0.1 to about 2 wt. %, about 0.1 to about 1 wt. %; from about 0.5 to about 7 wt. %, about 0.5 to about 5 wt. %, about 0.5 to about 4 wt. %, about 0.5 to about 3 wt. %, about 0.5 to about 2 wt. %, about 0.5 to about 1 wt. %; from about 1 to about 7 wt. %, about 1 to about 5 wt. %, about 1 to about 4 wt. %, about 1 to about 3 wt. %, about 1 to about 2 wt. %; from about 2 to about 7 wt. %, about 2 to about 5 wt. %, about 2 to about 4 wt. %, about 2 to about 3 wt. %; from about 3 to about 7 wt. %, about 3 to about 5 wt. %, about 3 to about 4 wt. %, or any range or subrange thereof, based on the total weight of the pet shampoo.

Thickening agents may be referred to as “thickeners” or “viscosity modifying agents.” Thickening agents are typically included to increase the viscosity of the pet shampoos. Non-limiting examples of thickening agents include natural gums, polyacrylate cross polymers or crosslinked polyacrylate polymers, cationic acrylate copolymers, anionic acrylic or carboxylic acid polymers, polyacrylamide polymers, polysaccharides such as cellulose derivatives, cationic gums, polyquaterniums, vinylpyrrolidone homopolymers/copolymers, C₈₋₂₄ hydroxyl substituted aliphatic acid, C₈₋₂₄ conjugated aliphatic acid, sugar fatty esters, polyglyceryl esters, and a mixture thereof. Particular types of thickening agents that may be mentioned include the following: gums (e.g., natural gums), sucrose esters, polyvinylpyrrolidone (PVP) and co-polymers, celluloses, polyquaternium compounds, and carboxylic acid or carboxylate based homopolymer or co-polymer, which can be linear or crosslinked.

In at least one embodiment, the pet shampoo comprises a polysaccharide, such as those selected from cellulose, xanthan gum, diutan gum, carrageenan, gellan gum, welan gum, pectin, sclerotium gum, starch, galactoarabinan, and a combination of two or more thereof.

The pet shampoos may in some cases comprise one or more builder(s). The builders may be selected from those based on renewable raw materials that can be obtained from plants, such as polyaspartates, polycarboxylates such as citrates, and gluconates, succinates or malonates. In at least one embodiment, the pet shampoo includes sodium gluconate. The total amount of builder(s) in the pet shampoo may be from about 0.1 to about 5 wt. %, about 0.1 to about 4 wt. %, about 0.1 to about 3 wt. %, about 0.1 to about 2 wt. %, about 0.1 to about 1 wt. %; from about 0.3 to about 5 wt. %, about 0.3 to about 4 wt. %, about 0.3 to about 3 wt. %, about 0.3 to about 2 wt. %, about 0.3 to about 1 wt. %; from about 0.6 to about 5 wt. %, about 0.6 to about 4 wt. %, about 0.6 to about 3 wt. %, about 0.6 to about 2 wt. %, about 0.6 to about 1 wt. %; from about 1 to about 5 wt. %, about 1 to about 4 wt. %, about 1 to about 3 wt. %, about 1 to about 2 wt. %; from about 1.5 to about 5 wt. %, about 1.5 to about 4 wt. %, about 1.5 to about 3 wt. %; from about 2 to about 5 wt. %, about 2 to about 4 wt. %, about 2 to about 3 wt. %; from about 3 to about 5 wt. %, about 3 to about 4 wt. %, about 4 to about 5 wt. %, including any range or subrange thereof, based on the total weight of the pet shampoo.

As mentioned above, the pet shampoo may be adapted to enhance the benefits of a pet food composition for reducing alopecia, skin secretions, car secretions, and/or erythema scores.

The pet food composition may comprise a blend of fats and oils, whole egg, antioxidants and sources of polyphenols formulated to help modulate the immune response to allergens. The functional goal of the inclusion of these nutrients is to help stabilize mast and inflammatory cells, thereby decrease histamine and cytokine release. Without being limited to any particular theory, it is believed that certain components within eggs provide immunomodulation and anti-oxidant activity. The polyphenols may be selected from flavonoids.

The pet food composition may include plant-based foods such as green tea, fruits, some herbs and green vegetables, which may be rich in polyphenols and/or antioxidants.

A combination of vitamin E, antioxidants, and omega-3 fatty acids may be included in certain pet food compositions. Vitamin E plays a vital role in protecting skin lipids from peroxidation secondary to exposure to UV radiation, inflammation or infection. Dietary alpha tocopherol increases serum vitamin E levels and serum vitamin E correlates with skin levels of vitamin E. The pet food composition may contain vitamin E in excess of the above recommended amount and also contains other antioxidant sources including vitamin C, green tea as source of catechins and lipoic acid.

The pet food compositions may, in certain embodiments, comprise a complex balance of macro and micronutrients to enhance healthy skin and hair coat. For instance, the pet food composition may comprise protein, Vitamins E, omega-3 and omega-6 fatty acids in certain ratios to one another. In certain embodiments, the pet food composition may also comprise minerals, such as zinc and copper, which when incorporated into formulas of the pet food composition may further promote growth and healing of the epidermis, dermis, collagen and pigmentation. The essential fatty acids, cis-linoleic and alpha-linolenic, which are omega-6 and omega-3 fatty acid families respectively, may be included. Omega-9 fatty acids may, additionally or alternatively, be included for improving signaling cascades that affect inflammation and cell growth.

The combination of nutrients in the pet food, according to an embodiment of the invention, includes (all percentages express weight percent based on total dry food weight, unless otherwise specified): Total dietary fat levels from sources like chicken, pork, and/or beef fat, of from 8% to 25%, from 10% to 20%, from 11% to 19%, from 12% to 18%, from 13% to 17%, from 14% to 16%; Omega 3, 6 and 9 fatty acids are present in a ratio of from about 0.5:1:1 to about 0.5:5:5, from about 0.5:1.5:1.5 to about 0.5:4:4, from about 0.5:1:1 to about 3:5:5, from about 0.5:1.5:1.5 to about 3:4:4, from 0.5:2.0:2.0 to 3.0:5.0:5.0, from 0.7:2.0:2.0 to 1.2:2.0:2.0; optionally about 0.8:2.0:2.0 to 1.1:2.0:2.0, optionally about 0.9:2.0:2.0 to 1.0:2.0:2.0. Without being limited to any particular theory, it is believed that certain ratios of the omega-3 fatty acids, omega-3 fatty acids, and omega-3 fatty acids may promote benefits to the hair and skin of a pet, which can be further enhanced by pet shampoos adapted enhance such benefits.

The fatty acids may come from sources like soybean oil, flaxseed, chicken fat, pork fat, beef fat, olive oil, sunflower oil, corn oil, coconut oil, and/or fish oil. Fatty acids are long carbon chains with a methyl group at one end. Polyunsaturated fatty acids have multiple double bonds. The fatty acids that have the first double bond three carbons away from the methyl group are the omega-3 (or n-3) series. The omega-6 (or n-6) series of polyunsaturated fatty acids have the first double bond six carbons from the methyl group. In mammals, polyunsaturated omega-3 and omega-6 fatty acids are essential fatty acids (“EFAs”) because they cannot be synthesized de novo by mammals. Members of the omega-6 family include, e.g., linoleic (18:211-6), gamma-linolenic (18:311-6), dihomogammalinolenic (20:311-6), and arachidonic acid (20:411-6). Members of the omega-3 family include, e.g., alpha-linolenic (18:3n−3), eicosatetraenoic (20:4n−3), cicosapentaenoic (20:5n−3), and docosahexaenoic acid (22:6n−3). In some embodiments, the pet food composition comprises an amount of linoleic acid that is equal to or higher than the minimum allowance recommended by the Association of American Feed Control Officials (“AAFCO”). In some embodiments, the composition comprises an amount of linoleic acid that is up to about 200% higher, up to about 300% higher, up to about 400% higher, up to about 500% higher, up to about 600% higher, up to about 700% higher, up to about 800% higher, up to about 900% higher, up to about 1000% higher, or up to about 1100% higher than the respective AAFCO minimum allowance. The AAFCO's minimum linoleic acid allowance for growth, reproduction, and maintenance of dogs is 1 wt. % on a dry matter basis (presuming that the energy density of the diet is 3.5 kcal ME/g dry matter). Therapeutic non-zero amounts of various ingredients can generally be determined by reference to such dietary guidelines and other materials within the general knowledge of food scientists and veterinary practitioners.

In some embodiments, the amount of egg product, such as dried egg present is from 0.1 to 15 wt. %, from 0.2 to 14 wt. %, from 0.3 to 13 wt. % from 0.4 to 12 wt. %, from 0.5 to 11 wt. %, from 0.7 to 10 wt. %, from 0.8 to 9 wt. %, from 0.9 to 8 wt. %, from 1 to 7 wt. %, from 2 to 6 wt. %, from 3 to 5 wt. %; the amount of Vitamin E present is from 200 IU/kg to 1,300 IU/kg, from 300 IU/kg to 1,200 IU/kg, 400 IU/kg to 1,100 IU/kg, from 500 IU/kg to 1,000 IU/kg, from 600 IU/kg to 800 IU/kg; the amount of Vitamin C present is from 30 ppm to 750 ppm, from 50 ppm to 700 ppm, from 75 ppm to 600 ppm, from 100 ppm to 500 ppm; the amount of dietary polyphenol, such as quercetin present is from 1.5 ppm to 50 ppm, from 3 ppm to 40 ppm, from 4 ppm to 30 ppm, from 5 ppm to 25 ppm, from 8 ppm to 20 ppm, and/or from 10 ppm to 15 ppm.

The pet food compositions preferably comprise sources of quercetin, such as green tea extract, pea powder, apple powder, cranberry powder, carrot powder, broccoli powder, rosemary extract, citrus extract, schinopsis extract, eugenia extract, and curcuma extract.

The food composition may be in the form of dry and wet (loaf, minced, stew) compositions, including nutritionally complete diets, snacks, and treats.

In another aspect, provided is an article of manufacture, such as for example a kit comprising elements which can be assembled to form the composition. In some embodiments, the kit comprises in separate containers in a single package or in separate containers in a virtual package, as appropriate a composition of the present invention or two or more ingredients, that, when combined together and optionally with additional ingredients that are not a part of the kit, yield a composition comprising one or more omega-3 fatty acids, one or more omega-6 fatty acids, and one or more omega-9 fatty acids, in relative amounts to produce a total omega-3 to total omega-6 fatty acid to total omega-9 fatty acid ratio of from about 0.9:2.0:2.0 to about 3.0:5.0:5.0, and a total omega-6 fatty acid content of from about 1.5 to about 12 wt. %; from 1 wt. % to 12 wt. % alpha-lipoic acid; from 0.1 wt. % to 15 wt. % of egg product; from 200 IU/kg to 1,300 IU/kg Vitamin E; from 30 ppm to 750 ppm Vitamin C; and from 1.5 ppm to 50 ppm polyphenol; wherein all percentages by weight are based on total dry weight. The kit may also include (1) instructions for feeding the composition to an animal, (2) instructions for making a composition of the present invention by combining the two or more ingredients, (3) one or more agents for managing non-food allergies, (4) instructions for administering the agents in conjunction with the composition, or (5) or a combination thereof. The term “single package” generally means that the components of a kit are physically associated in or with one or more containers and considered as a unit of manufacture, distribution, sale, or use.

In some embodiments, the pet food composition comprises one or more omega-3 fatty acids, one or more omega-6 fatty acids, and one or more omega-9 fatty acids, in relative amounts to produce a total omega-3 to total omega-6 fatty acid to total omega-9 fatty acid ratio of from about 0.5:2.0:2.0 to about 3.0:5.0:5.0, and a total omega-6 fatty acid content of from about 1.5 wt. % to about 12 wt. %; from 0.1 wt. % to 15 wt. % of egg product; and alpha-lipoic acid; polyphenol; Vitamin C and Vitamin E are present in therapeutic non-zero amounts, wherein all percentages by weight are based on total dry weight.

In further embodiments, the pet food composition comprises one or more omega-3 fatty acids, one or more omega-6 fatty acids, and one or more omega-9 fatty acids, in relative amounts to produce a total omega-3 to total omega-6 fatty acid to total omega-9 fatty acid ratio of from about 0.9:2.0:2.0 to about 3.0:5.0:5.0, and a total omega-6 fatty acid content of from about 1.5 wt. % to about 12 wt. %; from 0.01 wt. % to 0.12 wt. % alpha-lipoic acid; from 0.1 wt. % to 15 wt. % of egg product; from 200 IU/kg to 1,300 IU/kg Vitamin E; from 30 ppm to 750 ppm Vitamin C; and from 1.5 ppm to 50 ppm polyphenol; wherein all percentages by weight are based on total dry weight.

In additional embodiments, provided a method for managing allergies to non-food allergens in a companion animal comprising feeding the animal during an induction period prior to exposure of the animal to the non-food allergen with a food composition comprising: one or more omega-3 fatty acids, one or more omega-6 fatty acids, and one or more omega-9 fatty acids, in relative amounts to produce a total omega-3 to total omega-6 fatty acid to total omega-9 fatty acid ratio of from about 0.9:2.0:2.0 to about 3.0:5.0:5.0, and a total omega-6 fatty acid content of from about 1.5 wt. % to about 12 wt. %; from 0.01 wt. % to 0.12 wt. % alpha-lipoic acid; from 0.1 wt. % to 15 wt. % of egg product; from 200 IU/kg to 1,300 IU/kg Vitamin E; from 30 ppm to 750 ppm Vitamin C; and from 1.5 ppm to 50 ppm polyphenol; wherein all percentages by weight are based on total dry weight.

In yet further embodiments, provided is a kit for managing non-food allergies in a companion animal comprising two or more ingredients that, when combined together and optionally with additional ingredients that are not a part of the kit, yield a composition that comprises: one or more omega-3 fatty acids, one or more omega-6 fatty acids, and one or more omega-9 fatty acids, in relative amounts to produce a total omega-3 to total omega-6 fatty acid to total omega-9 fatty acid ratio of from about 0.9:2.0:2.0 to about 3.0:5.0:5.0, and a total omega-6 fatty acid content of from about 1.5 wt. % to about 12 wt. %; from 0.01 wt. % to 0.12 wt. % alpha-lipoic acid; from 0.1 wt. % to 15 wt. % of egg product; from 200 IU/kg to 1,300 IU/kg Vitamin E; from 30 ppm to 750 ppm Vitamin C; and from 1.5 ppm to 50 ppm polyphenol; wherein all percentages by weight are based on total dry weight.

In other embodiments, provided is a use of a pet food composition that comprises: one or more omega-3 fatty acids, one or more omega-6 fatty acids, and one or more omega-9 fatty acids, in relative amounts to produce a total omega-3 to total omega-6 fatty acid to total omega-9 fatty acid ratio of from about 0.9:2.0:2.0 to about 3.0:5.0:5.0, and a total omega-6 fatty acid content of from about 1.5 wt. % to about 12 wt. %; from 0.01 wt. % to 0.12 wt. % alpha-lipoic acid; from 0.1 wt. % to 15 wt. % of egg product; from 200 IU/kg to 1,300 IU/kg Vitamin E; from 30 ppm to 750 ppm Vitamin C; and from 1.5 ppm to 50 ppm polyphenol; wherein all percentages by weight are based on total dry weight to prepare a food for managing non-food allergies in a companion animal.

In some embodiments, the method steps of feeding a pet food composition to the pet and applying the pet shampoo to the pet can be performed in any order.

### EXAMPLES

### Example 1

A non-limiting example pet food composition (Example Pet Food Composition) was obtained in accordance with the formulation provided in Table 1.

TABLE 1

| | Example Pet Food |
|---|---|
| Description | Composition (wt. %) |
| Rice, brown | 45-50 |
| Rice, brewers | 10-20 |
| Egg Dried pelleted | 5-10 |
| Rice Protein, Conc, pwdr, IP | 2-7 |
| Flax seed whole brown | 1-5 |
| Dicalcium phosphate | 0.1-2 |
| Potassium chloride | 0.1-2 |
| Calcium carbonate | 0.1-1 |
| Fruit and Vegetable Blend, Silva | 0.1-1 |
| Lipoic acid, alpha, 5% | 0.1-1 |
| Vitamin E, adsorbate, 50% | 0.1-1 |
| Vitamin Premix M0619 | 0.1-1 |
| Choline chloride, liquid, 70% | 0.05-0.5 |
| Sodium chloride, iodized | 0.1-1 |
| Taurine | 0.05-0.5 |
| Mineral, premix, 2305 | 0.01-0.1 |
| Antioxidant, Verdilox OP | 0.01-0.1 |
| Natural Flavor | 0.01-0.1 |
| Beet, pulp, pelleted | 1-5 |
| Methionine, dl | 0.05-0.5 |
| Lactic acid, blend 84% | 0.5-5 |
| Soybean Oil, Refined, Naturally | 1-10 |
| Preserved | |
| PAL, C17007 | 1-5 |
| Fish oil, TG, 18/12, NP | 1-5 |
| Coconut oil preserved | 0.5-5 |

### Example 2

A non-limiting example pet shampoo (Example Pet Shampoo) was obtained in accordance with the formulation provided in Table 2.

TABLE 2

| | | |
|---|---|---|
| US INCI Compound Name | Example | Pet |
| | Shampoo | |
| | (wt. %) | |
| Decyl Glucoside | 5-20 | |
| Cocamidopropyl betaine | 1-10 | |
| Sodium Gluconate w/ Sodium Lactate | 1-5 | |
| Glycerin | 1-10 | |
| Flaxseed Oil | 0.1-1 | |
| Sodium Chloride | 0.1-1 | |
| Fragrance | 0.1-1 | |
| Xanthan Gum | 0.1-1 | |
| Citric Acid | 0.2-0.9 | |
| Tetrasodium EDTA | 0.01-0.2 | |
| Phenoxyethanol | 0.1-5 | |
| Water | Q.S. to 100 | |

### Example 3

A method for enhancing the reduction of alopecia, skin secretion, car secretion, and/or erythema scores of a pet by feeding the pet a non-limiting example pet food composition (Example Pet Food Composition) of Example 1 in combination with applying the non-limiting example pet shampoo (Example Pet Shampoo) of Example 2 was evaluated. Specifically, 13 dogs aging from 3 years to 12 years and 4 months and body weight ranging from 7.7 to 15.9 kg, which were all clinically diagnosed with CAD (canine atopic dermatitis) were used for the evaluation. The 13 dogs were of mixed gender and were neutered and spayed.

The dogs were pre-fed with an adult maintenance food for 28 days and divided into two groups by taking into account age, gender, and phenotype parameters (e.g., dermatological symptom severity). Group 1 consists of six dogs and Group 2 consists of seven dogs. After the pre-feed regimen, both groups underwent a feeding regimen where all of the dogs were fed the non-limiting Example Pet food Composition for a period of 35 days. The only difference between the two groups was the bathing condition. The dogs of group 1 were washed with only tap water (Control Group) and the dogs of Group 2 (Example Group) were washed with the Example Pet Shampoo followed by rinsing with tap water. Both groups of dogs were washed at the end of the pre-feed regimen and in the middle of the feeding regimen.

Dermatological health and symptoms were assessed by a veterinarian using a prescribed questionnaire equivalent to CADESI-4 at the end of pre-feeding regimen (“P1”), 48 hours after the first bath (“T3”), before the bath in the middle of the feeding regimen (“T21”), after the bath in the middle of the feeding regimen (“T24”), and after the end of the feeding regimen (“T35”). Another questionnaire was assessed by Animal Care Research Technicians (ACRTs), equivalent to pet parent observations at the end of pre-feeding regimen (“P1”), 24 hrs after the first bath in the middle of the feeding regimen (“T1”), before the second bath in the middle of the feeding regimen (“T21”), 24 hrs after the second bath in the middle of the feeding regimen (“T22”), and after the end of the feeding regimen (“T35”).

Other clinical endpoints such as skin pH and trans epidermal water loss (TEWL) were also measured at the end of pre-feeding regimen (“P1”), 48 hours after the first bath (“T3”), before the bath in the middle of the feeding regimen (“T21”), after the bath in the middle of the feeding regimen (“T24”), and after the end of the feeding regimen (“T35”). The skin pH of the dogs was measured in two body spots, specifically the right and left inguinal, and skin TEWL was measured in three body spots, namely the navel, right and left inguinal.

All 13 dogs with clinically diagnosed CAD were included for the questionnaire-based assessment analyses. Veterinarian questionnaire categorical data was converted into numerical data before calculating the improvement in the percentage of canines compared with the baseline for each group to alleviate CADsymptoms under four categories: pruritus, erythema, alopecia, and skin and car secretions. There are a set of questions answered in each category by the veterinarian based on his/her assessment of each dog and measured as none, mild, moderate and severe symptoms. These categorical measurements were converted into numerical data as described: “none”=0, “mild”=1, “moderate”=2 and “severe”=3. These measurements then were summed in each category by animal and by treatment to calculate the percent of canine improvement under these four categories by comparing results assessed at the end of the feeding regimen with the baseline, namely the assessment at the end of the pre-feeding regimen.

The ACRTs questionnaire categorical data was analyzed as a categorical data by calculating the delta (percent of canines improved) in each category (licking of paws, armpits, groin & anal area, scratching of cars, head shaking, skin redness & oily skin) as short term and long term efficacy of the Example Pet Shampoo. Short term efficacy was assessed by calculating the delta of the results from T1 minus P1 (T1−P1) and calculating the delta of the results T22 minus T21 (T22−T21). Long term efficacy was assessed by calculating the delta of the results T35 minus P1 (T35−P1).

Skin TEWL measurements were assessed by calculating the delta of the results of T3 minus P1 (T3−P1) and calculating the delta of the results of T24 minus T21 (T24−T21). Skin pH was analyzed by combining all measurements and compared between the control group versus the group receiving the Example Pet Shampoo.

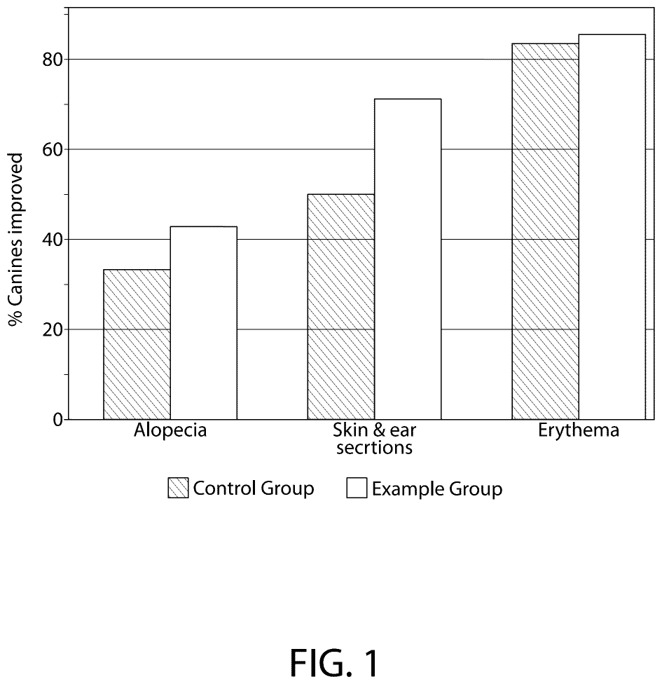

The combination of the Example Pet Food Composition and the Example Pet Shampoo enhanced the reduction of alopecia, skin and car secretions, and erythema scores, by increasing the response rate (improvement %) of dogs to 42.85%, 71.42%, and 85.71% respectively when compared with the control group (see FIG. 1). Alopecia (hair loss) can occur due to itchiness and persistent scratching specifically in the dogs with dermatological problems.

In general, dogs with CAD show a decrease in the quality of life due to behavioral patterns such as licking of body parts, car scratching, head shaking and skin redness due to the itchiness. Here, the combination of the Example Pet Food Composition and the Example Pet Shampoo increased the improvement % of dogs by reducing those behavioral patterns such as licking body parts showed improvement by 42.85%, ear scratching by 28.57%, head shaking by 14.29% and skin redness by 42.86% compared with the control group (see Table 3).

TABLE 3

| | Short term efficacy | | | | Long term efficacy | |
|---|---|---|---|---|---|---|
| | First bath (delta = | | Second bath | | End of the study (delta = | |
| | T1 − P1) | | (delta = T22 − T21) | | T35 − P1) | |
| % improvement (delta) | Control | Test | Control | Test | Control | Test |
| Licking of paws, armpits, | 0 | 14.3 | 50 | 14.3 | 33.3 | 42.9 |
| groin & anal area | | | | | | |
| Scratching of ears | 0 | 14.3 | 50 | 42.9 | 16.7 | 28.6 |
| Head shaking | 0 | 0 | 50 | 42.9 | 0 | 14.3 |
| Skin redness | 0 | 0 | 0 | 28.6 | 33.3 | 42.9 |
| Oily skin | 0 | 14.3 | 33.3 | 42.9 | 0 | 0 |

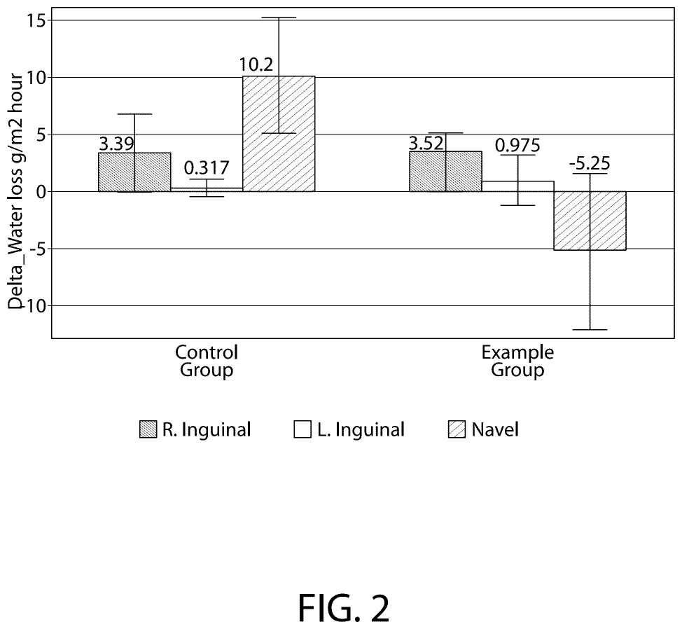

Further, the combination of the Example Pet Food Composition and the Example Pet Shampoo increased the improvement % of dogs by reducing the oily skin condition as a short term effect immediately after the first bath by 14.28% and increased to 42.86% after the second bath (as seen in Table 3) compared with the control group. In addition, we measured the trans epidermal water loss (TEWL) as a marker for skin membrane barrier function and observed no differences between control and test groups in two body spots (left and right inguinal). However the third body spot (navel) showed decreasing trend in the test group compared with the control group which suggests that the combination of the Example Pet Food Composition and the Example Pet Shampoo shows some improvement in skin membrane barrier function in the specific spot but not necessarily all body spots measured in this Example (FIG. 2). Also, no differences were observed in skin pH between control and test groups and all were considered to be in a healthy range.

### Example 4

A consumer sensory evaluation was conducted for Example Pet Shampoo. Specifically, 45 volunteers were given questionnaires evaluating various sensory aspects of a sample of the Example Pet Shampoo.

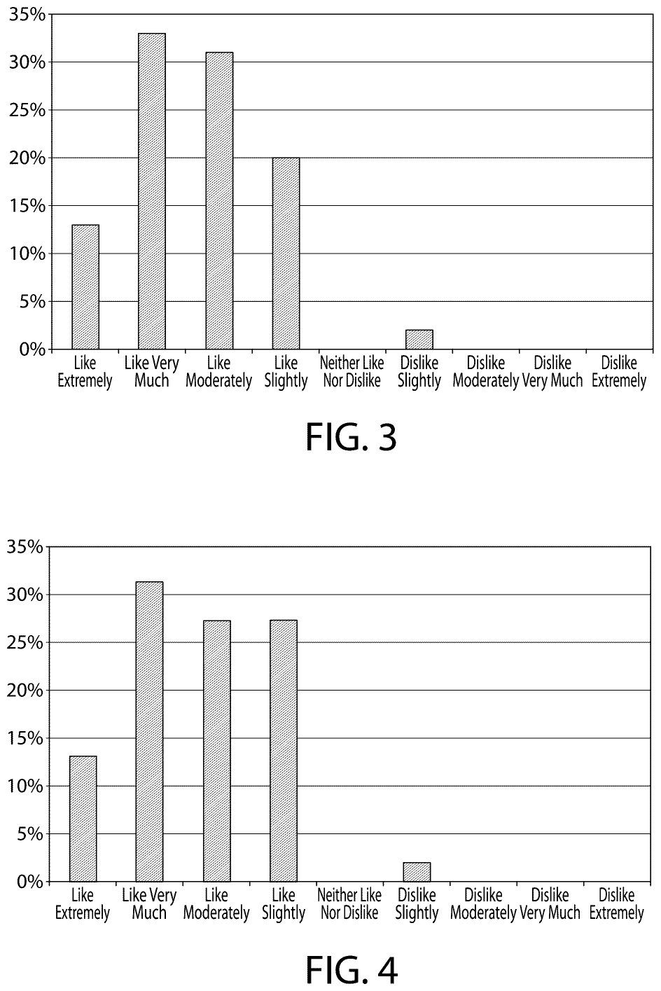

A summary of the “Aroma” results for a sample of the Pet Shampoo is shown in FIG. 3. FIG. 4 provides a summary of the results of the “Overall likeness” of a sample of the Pet Shampoo. Additionally, when asked if they would use the shampoo on their own dog, the majority of consumers said yes with only 9 out of the 45 saying no.

What is claimed is:

1. A method for improving the health and/or appearance of a pet's skin and/or hair, the method comprising:
feeding a pet food composition to a pet, the pet food composition comprising:
a plurality of fatty acids selected from omega-3 fatty acids, omega-6 fatty acids, omega-9 fatty acids, and a combination of two or more thereof;
quercetin; and
one or more vitamins, the one or more vitamins comprising a vitamin C, a vitamin E, or a combination thereof; and
applying a pet shampoo to the pet within about 36 hours of providing the pet food composition to the pet, the pet shampoo comprising:
from about 1 to about 15 wt. % of an amphoteric surfactant;
from about 5 to about 25 wt. % of a nonionic surfactant; and
from about 1 to about 20 wt. % of a polyol.

2. The method according to claim 1 further comprising:
rinsing the pet after applying the pet shampoo to the pet, wherein the pet is rinsed after a period of time ranging from about 30 seconds to about 30 minutes after the pet shampoo is applied.

3. The method according to claim 1 comprising reducing alopecia by about 20% or more.

4. The method according to claim 1 comprising reducing the skin secretions and/or the ear secretions of the pet by about 40% or more.

5. The method according to claim 1 comprising reducing erythema by about 50% or more.

6. The method according to claim 1, wherein the amphoteric surfactant is selected from the group consisting of alkyl amphopropionates, betaines, alkyl sultaines, alkyl amphoacetates, and a combination of two or more thereof.

7. The method according to claim 6, wherein the amphoteric surfactant comprises cocamidopropyl betaine.

8. The method according to claim 1, wherein the nonionic surfactant comprises a glucoside surfactant selected from the group consisting of decyl glucoside, caprylyl/capryl glucoside, caprylyl glucoside, lauryl glucoside, coco-glucoside, octyl glucoside, cetearyl glucoside, cetyl glucoside, hexadecyl glucoside, arachidyl glucoside, and a combination of two or more thereof.

9. The method according to claim 1, wherein the pet shampoo has a weight ratio of the amphoteric surfactant to the nonionic surfactant of about 1:1.5 to about 1:5.

10. The method according to claim 1, wherein the polyol is selected from glycerin, 1,3-propanediol, isoprene glycol, pentylene glycol, hexylene glycol, ethylene glycol, propylene glycol, butylene glycol, diethylene glycol, dipropylene glycol, and a combination of two or more thereof.

11. The method according to claim 1, wherein the pet shampoo further comprises an oil selected from the group consisting of flax seed oil, amaranth seed oil, apricot kernel oil, argan oil, avocado oil, babassu oil, cottonseed oil, borage seed oil, camelina oil, thistle oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, marula oil, evening primrose oil, olive oil, palm oil, palm kernel oil, Brazil nut oil, pecan nut oil, peach kernel oil, rapeseed oil, castor oil, sea buckthorn pulp oil, sea buckthorn kernel oil, sesame oil, soy bean oil, sunflower oil, grape seed oil, walnut oil, wild rose oil, wheat germ oil, coconut oil, and a combination of two or more thereof.

12. The method according to claim 1, wherein the pet shampoo is substantially free, or free, of an anionic surfactant.

13. The method according to claim 1, wherein the plurality of fatty acids comprises one or more omega-3 fatty acid, one or more omega-6 fatty acid, and one or more omega-9 fatty acid.

14. The method according to claim 13, wherein the pet food composition has a ratio of the total amount of the one or more omega-3 fatty acid to the total amount of the one or more omega-6 fatty acid to the total amount of the one or more omega-9 fatty acid in an amount ranging from about 0.5:2:2 to about 3:5:5.

15. The method according to claim 1, wherein the one or more vitamins comprises a vitamin C and a vitamin E.

16. The method according to claim 1, wherein the pet shampoo further comprises sodium gluconate.

17. The method according to claim 1, further comprising reducing the number of times the pet licks itself by about 40% or more.

18. The method according to claim 1, further comprising reducing the amount of ear scratching by the pet by about 20% or more.

19. A pet shampoo comprising:
an amphoteric surfactant;
a nonionic surfactant,
wherein the amphoteric surfactant and the nonionic surfactant are in a weight ratio of amphoteric surfactant to nonionic surfactant of about 1:1.5 to about 1:5; and
a polyol,
wherein the pet shampoo enhances the skin benefits provided by a pet food composition.

20. A method for improving the health and/or appearance of a pet's skin and/or hair, the method comprising:
applying a pet shampoo to a pet, the pet shampoo comprising:
from about 1 to about 15 wt. % of an amphoteric surfactant;
from about 5 to about 25 wt. % of a nonionic surfactant; and
from about 1 to about 20 wt. % of a polyol; and
feeding a pet food composition to the pet within about 36 hours of applying the pet shampoo to the pet, the pet food composition comprising:
a plurality of fatty acids selected from omega-3 fatty acids, omega-6 fatty acids, omega-9 fatty acids, and a combination of two or more thereof;
quercetin; and
one or more vitamins, the one or more vitamins comprising a vitamin C, a vitamin E, or a combination thereof.
